(19) United States
(12) Patent Application Publication
(10) Pub. No.: US 2010/0233268 A1
(43) Pub. Date: Sep. 16, 2010

(54) POWDER FORMULATIONS FOR INHALATION CONTAINING ENANTIOMERICALLY PURE BETA-AGONISTS

(75) Inventors: Michael Trunk, Ingelheim (DE); Marc Egen, Ingelheim (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/377,919

(22) PCT Filed: Aug. 21, 2007
(86) PCT No.: PCT/EP07/58651

§ 371 Date: Mar. 27, 2009

(30) Foreign Application Priority Data
Aug. 22, 2006 (EP) 06119270.4

(51) Int. Cl. A61K 9/14; C07D 265/18; A61K 31/538; C07D 265/12; A61P 11/00; A61P 11/06

(52) U.S. Cl. 424/489; 544/92; 514/230.5; 544/71

(57) ABSTRACT

The present invention relates to powder formulations for inhalation containing enantiomerically compounds of general Formula (I) wherein the groups R¹, R² and R³ may have the meanings indicated in the claims and in the specification, optionally in the form of the pharmaceutically acceptable acid addition salts thereof, and optionally in combination with a pharmaceutically acceptable excipient, processes for preparing them and their use as medicaments, particularly as medicaments for the treatment of respiratory complaints.

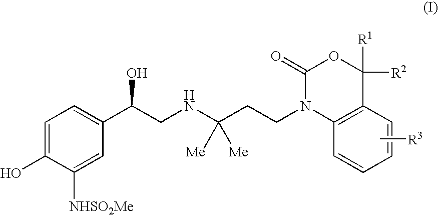

The present invention relates to powder formulations for inhalation, containing enantiomerically pure compounds of general formula 1

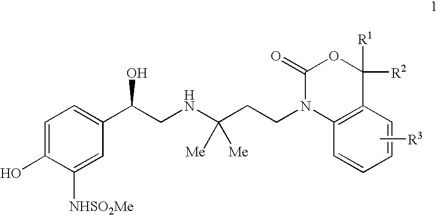

wherein the groups R¹, R² and R³ may have the meanings indicated in the claims and in the specification, optionally in the form of the pharmaceutically acceptable acid addition salts thereof, and optionally in combination with a pharmaceutically acceptable excipient, processes for preparing them and their use as medicaments, particularly as medicaments for the treatment of respiratory complaints.

### BACKGROUND TO THE INVENTION

Betamimetics (β-adrenergic substances) are known from the prior art. In this respect reference may be made for example to the disclosure of U.S. Pat. No. 4,460,581 which proposes betamimetics for the treatment of a wide range of ailments.

For drug treatment of diseases it is often desirable to prepare medicaments with a longer duration of activity. As a rule, this ensures that the concentration of the active substance in the body needed to achieve the therapeutic effect is maintained for a longer period without the need to re-administer the drug at frequent intervals. Moreover, giving an active substance at longer time intervals contributes to the well-being of the patient to a high degree.

In a particularly preferred embodiment the present invention relates to medicament preparations that may confer a therapeutic benefit in the treatment of respiratory complaints.

For treating respiratory complaints, in particular, it is useful to administer the active substance by inhalation. In addition to the administration of broncholytically active compounds in the form of metered aerosols and inhalable solutions, the use of inhalable powders containing active substance is of particular importance.

With active substances which have a particularly high efficacy, only small amounts of the active substance are needed per single dose to achieve the desired therapeutic effect. In such cases, the active substance has to be diluted with suitable excipients in order to prepare the inhalable powder. Because of the large amount of excipient, the properties of the inhalable powder are critically influenced by the choice of excipient. When choosing the excipient its particle size is particularly important. As a rule, the finer the excipient, the poorer its flow properties. However, good flow properties are a prerequisite for highly accurate metering when packing and dividing up the individual doses of preparation, e.g. when producing capsules (inhalettes) for powder inhalation, filling containers of different kinds, e.g. In a multi-dose powder inhaler with pre-filled doses, or when the patient is metering the individual dose before using a multi-dose inhaler. Moreover, the particle size of the excipient is very important for the emptying characteristics of capsules when used in an inhaler. It has also been found that the particle size of the excipient has a considerable influence on the proportion of active substance in the inhalable powder which is delivered for inhalation. The term inhalable proportion of active substance refers to the particles of the inhalable powder which are conveyed deep into the branches of the lungs when inhaled with a breath. The particle size required for this is between 0.5 and 10 μm, preferably between 1 and 6 μm.

The aim of the invention is to prepare an inhalable powder containing a betamimetic which, while being accurately metered (in terms of the amount of active substance and powder mixture packed into each powder charge by the manufacturer as well as the quantity of active substance released and delivered to the lungs on each application by the inhalation process) with only slight variations between batches, enables the active substance to be administered in a large inhalable proportion. A further aim of the present invention is to prepare an inhalable powder containing a betamimetic which ensures good emptying characteristics of the capsules, whether it is administered to the patient using an inhaler, for example, as described in WO 94/28958, or in vitro using an impactor or impinger.

The fact that betamimetics have a high therapeutic efficacy even at very low doses imposes further conditions on an inhalable powder which is to be used with highly accurate metering. Because only a low concentration of the active substance is needed in the inhalable powder to achieve the therapeutic effect, a high degree of homogeneity of the powder mixture and only slight fluctuations in the dispersion characteristics from one batch of powder to the next are essential. The homogeneity of the powder mixture and minor fluctuations in the dispersion properties are crucial in ensuring that the inhalable proportion of active substance is released reproducibly in constant amounts and with the lowest possible variability.

Accordingly, a further aim of the present invention is to prepare an inhalable powder containing a betamimetic which is characterised by a high degree of homogeneity and uniformity of dispersion. The present invention also sets out to provide an inhalable powder which allows the inhalable proportion of active substance to be administered with the lowest possible variability.

The characteristics of emptying from the powder reservoir (the container from which the inhalable powder containing the active substance is released for inhalation) play an important part, not exclusively, but especially in the administration of inhalable powders using capsules containing powder. If only a small amount of the powder formulation is released from the powder reservoir as a result of minimal or poor emptying characteristics, significant amounts of the inhalable powder containing the active substance are left in the powder reservoir (e.g. The capsule) and are unavailable to the patient for therapeutic use. The result of this is that the dosage of active substance in the powder mixture has to be increased so that the quantity of active substance delivered is sufficient to produce the desired therapeutic effect.

Against this background the present invention further sets out to provide an inhalable powder containing a betamimetic which is also characterised by very good emptying characteristics.

### DESCRIPTION OF THE INVENTION

The present invention relates to inhalable powders containing one or more, preferably one, enantiomerically pure compound of general formula 1

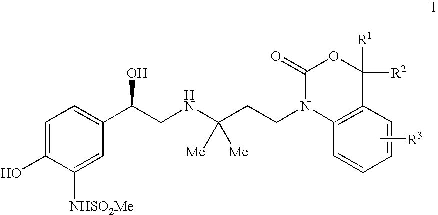

wherein
R¹ and R² independently of one another denote H, halogen or C₁₋₄-alkyl or together denote C₁₋₆-alkylene; and
R³ denotes H, halogen, OH, C₁₋₄-alkyl or O—C₁₋₄-alkyl;
   optionally in the form of the pharmaceutically acceptable acid addition salts, hydrates or solvates thereof, optionally in admixture with one or more physiologically acceptable excipients.

Preferred inhalable powders as mentioned hereinbefore are those which contain one or more, preferably one, enantiomerically pure compound of general formula 1, wherein
R¹ and R² which may be identical or different, denote hydrogen, fluorine, chlorine, methyl, ethyl, propyl, butyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, chlorine, OH, methyl, ethyl, methoxy, or ethoxy,
   optionally in the form of the pharmaceutically acceptable acid addition salts, hydrates or solvates thereof, optionally in admixture with one or more physiologically acceptable excipients.

Preferred inhalable powders are those that contain one or more, preferably one, enantiomerically pure compound of general formula 1, wherein
R¹ and R² which may be identical or different, denote hydrogen, methyl, ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy;
   optionally in the form of the pharmaceutically acceptable acid addition salts, hydrates or solvates thereof, optionally in admixture with one or more physiologically acceptable excipients.

Preferred inhalable powders are those that contain one or more, preferably one, enantiomerically pure compound of general formula 1, wherein
R¹ and R² which may be identical or different, denote ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy,
   optionally in the form of the pharmaceutically acceptable acid addition salts, hydrates or solvates thereof, optionally in admixture with one or more physiologically acceptable excipients.

Preferred inhalable powders are those that contain one or more, preferably one, enantiomerically pure compound of general formula 1, wherein
R¹ and R² represent ethyl, propyl or together represent —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH or methoxy,
   optionally in the form of the pharmaceutically acceptable acid addition salts, hydrates or solvates thereof, optionally in admixture with one or more physiologically acceptable excipients.

Also preferred according to the invention are inhalable powders containing one or more, preferably one, enantiomerically pure compound of general formula 1 in the form of the free bases thereof.

Of equivalent importance according to the invention are also inhalable powders that contain one or more, preferably one, enantiomerically pure compound of general formula 1 in the form of the pharmaceutically acceptable acid addition salts thereof which can be represented by general formula 1-HX.

Preferred inhalable powders contain as acid addition salts one or more, preferably one, compound of general formula 1-HX,

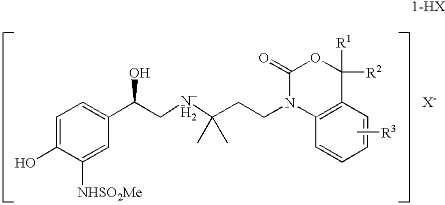

wherein
X⁻ denotes a mono- or polysubstituted negatively charged anion, preferably a mono- or polysubstituted negatively charged anion selected from among chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
   and the groups R¹, R² and R³ may have one of the meanings given above, optionally in the form of the tautomers, mixtures of tautomers, hydrates or solvates thereof, and optionally in admixture with one or more physiologically acceptable excipients.

Preferred inhalable powders contain one or more, preferably one, compound of formula 1-HX, wherein
X⁻ denotes a mono- or polysubstituted negatively charged anion selected from among chloride, bromide, sulphate, methanesulphonate, maleate, acetate, benzoate, citrate, salicylate, trifluoroacetate, fumarate, tartrate and succinate;
   and the groups R¹, R², R³ and R⁴ may have one of the meanings given above, optionally in the form of the tautomers, mixtures of tautomers, hydrates or solvates thereof, and optionally in admixture with one or more physiologically acceptable excipients.

Preferred inhalable powders contain one or more, preferably one, compound of formula 1-HX, wherein
X⁻ denotes a mono- or polysubstituted negatively charged anion selected from among chloride, methanesulphonate, maleate, acetate, citrate, salicylate, trifluoroacetate, fumarate and succinate, preferably chloride, maleate, salicylate, fumarate and succinate, particularly preferably chloride;
   and the groups R¹, R², R³ and R⁴ may have one of the meanings given above, optionally in the form of the tautomers, mixtures of tautomers, hydrates or solvates thereof, and optionally in admixture with one or more physiologically acceptable excipients.

Also particularly preferred are inhalable powders that contain one or more, preferably one, enantiomerically pure compound of general formula 1 selected from among
N-(5-{2-[1,1-dimethyl-3-(4-methyl-2-oxo-4H-benzo[d][1,3]oxazin-1-yl)-propylamino]-1-hydroxy-ethyl}-2-hydroxy-phenyl)-methanesulphonamide
N-(5-{2-[1,1-dimethyl-3-(2-oxo-4H-benzo[d][1,3]oxazin-1-yl)-propylamino]-1-hydroxy-ethyl}-2-hydroxy-phenyl)-methanesulphonamide
N-(5-{2-[3-(4-ethyl-2-oxo-4H-benzo[d][1,3]oxazin-1-yl)-1,1-dimethyl-propylamino]-1-hydroxy-ethyl}-2-hydroxy-phenyl)-methanesulphonamide
N-(5-{2-[3-(4,4-dimethyl-2-oxo-4H-benzo[d][1,3]oxazin-1-yl)-1,1-dimethyl-propylamino]-1-hydroxy-ethyl}-2-hydroxy-phenyl)-methanesulphonamide
N-(2-hydroxy-5-{1-hydroxy-2-[3-(6-hydroxy-4,4-dimethyl-2-oxo-4H-benzo[d][1,3]oxazin-1-yl)-1,1-dimethyl-propylamino]-ethyl}-phenyl)-methanesulphonamide
N-(2-hydroxy-5-{1-hydroxy-2-[3-(6-methoxy-4,4-dimethyl-2-oxo-4H-benzo[d][1,3]oxazin-1-yl)-1,1-dimethyl-propylamino]-ethyl}-phenyl)-methanesulphonamide
N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide
N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide
N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide
N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide:
N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide
N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide
N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide
N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide
   optionally in the form of the tautomers, mixtures of tautomers, hydrates or solvates thereof.

Also particularly preferred are inhalable powders that contain one or more, preferably one, enantiomerically pure compound of general formula 1 selected from among
N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide
N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide
N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide
N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide:
N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide
N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide
N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide
N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide,
   optionally in the form of the tautomers, mixtures of tautomers, hydrates or solvates thereof.

In the inhalable powders according to the invention the enantiomerically pure compounds of general formula 1, wherein R¹, R² and R³ have the above-mentioned meanings, are present in crystalline form, optionally in the form of the crystalline tautomers, crystalline hydrates or crystalline solvates thereof. Particularly preferred are enantiomerically pure, crystalline compounds of general formula 1 wherein R¹, R² and R³ have the above-mentioned meanings, optionally in the form of the crystalline tautomers, crystalline hydrates or crystalline solvates thereof, which are further characterised in that they are crystalline compounds that are present in only a single crystalline modification.

By the expression “a single crystalline modification” are meant crystalline compounds of formula 1 which are not a mixture of any existing polymorphous crystalline modifications and/or mixtures of one or more crystalline modifications with the amorphous or glassy state of the compounds according to formula 1.

### Terms and Definitions Used

By the term “C₁₋₄-alkyl” (including those which are part of other groups) are meant branched and unbranched alkyl groups with 1 to 4 carbon atoms. Examples include: methyl, ethyl, n-propyl, iso-propyl, n-butyl, iso-butyl, sec-butyl, tert-butyl, n-pentyl, iso-pentyl, neo-pentyl or hexyl. The following abbreviations may optionally also be used for the above-mentioned groups: Me, Et, n-Pr, i-Pr, n-Bu, i-Bu, t-Bu, etc. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₁₋₆-alkylene” (including those which are part of other groups) are meant branched and unbranched alkylene groups with 1 to 6 carbon atoms. Examples include: methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene or hexylene. Unless stated otherwise, the definitions propylene, butylene, pentylene and hexylene include all the possible isomeric forms of the groups in question with the same number of carbons. Thus, for example, propyl also includes 1-methylethylene and butylene includes 1-methylpropylene, 1,1-dimethylethylene, 1,2-dimethylethylene.

“Halogen” within the scope of the present invention denotes fluorine, chlorine, bromine or iodine. Unless stated to the contrary, fluorine, chlorine and bromine are regarded as preferred halogens.

The term enantiomerically pure within the scope of the present invention describes compounds of formula 1 which are present in an enantiomeric purity of at least 85% ee, preferably at least 90% ee, particularly preferably >95% ee. The term ee (enantiomeric excess) is known in the art and describes the optical purity of chiral compounds.

In order to prepare the inhalable powders according to the invention it is first necessary to make the compounds of formula 1 obtained in crystalline form into a finely divided (or micronised) form.

The micronisation or grinding process may be carried out using conventional mills. Preferably, the micronisation is carried out with the exclusion of moisture, more preferably, using a corresponding inert gas such as nitrogen, for example. It has proved particularly preferable to use air jet mills in which the material is comminuted by the impact of the particles on one another and on the walls of the grinding container. The micronisation may be carried out using both so-called countercurrent mills, optionally with a subsequent sifting process, or preferably using spiral air jet mills. According to the invention, nitrogen is preferably used as the grinding gas. The material for grinding is conveyed by the grinding gas under specific pressures (grinding pressure). Within the scope of the present invention, the grinding pressure is usually set to a value between about 2 and 8 bar, preferably between about 3 and 7 bar, most preferably between about 3.5 and 6.5 bar. The material for grinding is fed into the air jet mill by means of the feed gas under specific pressures (feed pressure). Within the scope of the present invention a feed pressure of between about 2 and 8 bar, preferably between about 3 and 7 bar and most preferably between about 3.5 and 6 bar has proved satisfactory. The feed gas used is also preferably an inert gas, most preferably nitrogen again. The material to be ground (crystalline compounds according to formula 1) may be fed in at a rate of about 5-45 g/min, preferably at about 15-35 g/min.

For example, without restricting the subject of the invention thereto, the following apparatus has proved suitable as a possible embodiment of an air jet mill: a 2-inch Microniser with grinding ring, 0.8 mm bore, made by Messrs Sturtevant Inc., 348 Circuit Street, Hanover, Mass. 02239, USA. Using the apparatus, the grinding process is preferably carried out with the following grinding parameters: grinding pressure: about 4.5-6.5 bar; feed pressure: about 4.5-6.5 bar; supply of grinding material: about 17-21 g/min.

Another example that may be mentioned is the use of an air jet mill made by Messrs Jetpharma, of the type Jetmill MC 50, which can be operated with the following process parameters:

| | | |
|---|---|---|
| grinding pressure: | 8.0 bar | (+/−0.5 bar) |
| feed pressure: | 8.5 bar | (+/−0.5 bar), |
| product supply: | 20 g/min | (+/−2.0 g/min) |
| jet setting (injector): | 37.2 mm | (constant) |

note:
the feed pressure is always set 0.25 to 0.5 bar higher than the grinding pressure

The ground material thus obtained may then be further processed under the following specific conditions. The micronisate is exposed to a water vapour at a relative humidity of at least 40% at a temperature of 15-50° C., preferably 20-45° C., most preferably 25-40° C. Preferably, the humidity is set to a value of 50-95% r.h., preferably to 60-90% r.h., most preferably 70-85% r.h.

By relative humidity (r.h.) is meant within the scope of the present invention the quotient of the partial steam pressure and the steam pressure of the water at the temperature in question. Preferably, the micronisate obtained from the grinding process described above is subjected to the chamber conditions mentioned above for a period of at least 6 hours. Preferably, however, the micronisate is subjected to the chamber conditions mentioned above for about 12 to about 120 hours, preferably about 15 to about 96 hours, particularly preferably about 18 to about 72 hours.

In one variant, this step is followed by after-drying. The ground material is exposed to an elevated temperature. In order to do this, the micronisate is subjected to an elevated temperature of at least 40° C., preferably at least 50° C. and at most 70° C. at reduced relative humidity, i.e. a relative humidity of less than 60%, preferably less than 40% and particularly preferably less than 30%, over a period of at least 0.5 hours, preferably 0.5 hours to 6 hours, particularly preferably 0.5 hours to 3 hours. The above drying process may optionally also be supplemented by the application of a vacuum.

In one variant it is also possible to further process the micronised material by subjecting it to a gaseous phase of an organic solvent at temperatures between 15° C.-45° C., preferably 20° C.-35° C. This step should last for at least 6 h, while periods of up to 12 h and also up to 24 h, and up to 48 h may be applied. In this step, the micronised material is subjected to a vapour pressure of at least 40%. Preferably the vapour pressure is set to a level of 50-100% r.h., preferably 60-99% r.h., particularly preferably 70-98% r.h. By the vapour pressure is meant within the scope of the present invention the quotient of the partial pressure of the gaseous phase of the solvent and the partial pressure of the gaseous phase of the organic solvent at the temperature in question.

In one variant this step is followed by a degassing step. The ground material is exposed to an elevated temperature. For this purpose, the micronised material is subjected to an elevated temperature of at least 40° C., preferably at least 50° C. and at most 70° C., over a period of at least 0.5 hours, preferably 0.5 hours to 6 hours, particularly preferably 0.5 hours to 3 hours, while continuously exchanging the gaseous phase directly above the ground material. This additional step can optionally be supplemented by the application of a vacuum. Suitable solvents for carrying out this step have proved to be non-polar solvents with a high vapour pressure (greater than that of the water) and a low boiling point (<150° C., preferably <120° C., most particularly preferably <100° C.). Examples of these, without any claim to naming them all, are ethanol, methanol, chloroform, methylene chloride, alkanes, such as e.g. pentane, hexane, heptane and cyclohexane.

The micronised compounds of formula 1 according to the invention that can be obtained by the above process have a characteristic particle size X₅₀ of between 0.1 μm and 10 μm, preferably between 0.5 μm and 6 μm, particularly preferably between 1.0 μm and 3.5 μm. In addition they are characterised by the parameter Q_{(5.8)} of more than 60%, preferably more than 70%, most preferably more than 80%.

The characteristic value Q_{(5.8)} indicates the median value of the particle size below which 50% of the particles are found, in relation to the volume distribution of the individual particles. The characteristic value Q_{(5.8)} corresponds to the quantity of particles which are below 5.8 μm, based on the volume distribution of the particles. The particle sizes were determined within the scope of the present invention by laser diffraction (Fraunhofer diffraction). The particle sizes were determined by laser diffraction (Fraunhofer diffraction) using the method described in WO 03/078429 (page 16 ff).

The micronised compounds of general formula 1 described above may optionally be administered by inhalation without any other excipient. Preferably, however, the pharmaceutical compositions according to the invention contain, in addition to one or more, preferably one, compound of formula 1, at least one physiologically acceptable excipient or a mixture of physiologically acceptable excipients. Examples of physiologically acceptable excipients which may be used to prepare the inhalable powders according to the invention include, for example, monosaccharides (e.g. glucose, fructose or arabinose), disaccharides (e.g. lactose, saccharose, maltose or trehalose), oligo- and polysaccharides (e.g. maltodextrin, starch, cellulose and the derivatives thereof), polylactide/glycolide (Resomer), polyalcohols (e.g. sorbitol, mannitol, xylitol), amino acids (arginine hydrochloride), chitosan (particularly preferably lactose, mannitol, saccharose, sorbitol, trehalose), alkali metal and alkaline earth metal salts of stearic acid (e.g. Mg stearate), salts (e.g. sodium chloride, calcium carbonate) or mixtures of these excipients with one another. Preferably, mono- or disaccharides or polyalcohols are used, while the use of lactose, glucose, trehalose or mannitol, preferably lactose, mannitol or glucose, is preferred, particularly, but not exclusively, in the form of their hydrates. For the purposes of the invention, lactose or mannitol is the particularly preferred excipient, while lactose monohydrate or mannitol is most particularly preferred. In another variant, formulations of the active substance with beta-lactose anhydrate and mixtures with a constant ratio of beta-lactose and alpha-lactose monohydrate are also included according to the invention.

In pharmaceutical formulations according to the invention which contain in addition to a compound of formula 1 a physiologically acceptable excipient, the ratio of the compound of formula 1 to the excipient is usually in the range from 5:100 to 1:100000, preferably 3:1000 to 1:10000 and particularly preferably from 3:1000 to 1:10000, the ratios given above being ratios by weight (w/w).

According to the invention another process step can be added on, which comprises drying the inhalable powder in the capsule, for example. By exposing the inhalable powder or the product packed into capsules to a relative humidity of between 10% r.h. and 50% r.h. (based on 25° C.), or alternatively between 10% and 40% r.h. (based on 25° C.), or alternatively between 10% and 30% r.h. (based on 25° C.), water is removed from the product. Preferably the product mentioned above is subjected to the above-mentioned climatic conditions for a period of at least 6 hours. Preferably the product is subjected to the above-mentioned climatic conditions for about 12 to about 120 hours, preferably about 15 to about 96 hours, particularly preferably about 18 to about 72 hours.

The inhalable powders according to the invention are usually administered in amounts of 3-100 mg, preferably 5-50 mg for each inhalation.

If the inhalable powders according to the invention do not contain any excipient, only one or more, preferably one compound of formula 1 in micronised form, 1 to 30 μg, preferably 3 to 100 μg and particularly preferably 5 to 520 μg inhalable powder are usually administered per inhalation.

The inhalable powders according to the invention are preferably administered in the form of a pre-metered pharmaceutical preparation. Examples include an inhalation capsule system. It is also possible to use systems wherein the powder preparation is presented in single doses e.g. contained in blister wells. In another form the preparations according to the invention are also suitable for use in inhalers which have a powder reservoir and wherein the quantity of powder to be administered or the crystalline micronisate of the active substance is not metered or divided up until immediately prior to use. The powder preparations described here may be inhaled by means of a suitable inhaler. Suitable inhalers are known from the prior art. Particularly suitable inhalers are mentioned for example in WO 03/084502, the contents of which are hereby incorporated by reference with regard to the inhalers disclosed therein.

The inhalable powders prepared according to the invention may be prepared as described below.

The process for preparing inhalable powders according to the invention is characterised in that N+m substantially equal portions of the physiologically acceptable excipient and N equal portions of the micronised compound of formula 1 are placed in alternate layers in a suitable mixing vessel and after they have all been added the 2N+m layers of the two components are mixed together using a suitable mixer, a portion of the physiologically acceptable excipient being put in first, while N is an integer >0, preferably >1, and m denotes 0 or 1.

Preferably, the individual fractions are added in layers through a suitable screening apparatus. If desired, once the mixing process is finished, the entire powder mixture can be subjected to one or more additional screening processes. In the process according to the invention, N is naturally dependent inter alia on the total quantity of powder mixture to be produced. When producing smaller batches, the desired effect of high homogeneity in the sense of uniformity of content can be achieved with a smaller N. In principle, however, it is preferable according to the invention if N is at least 10 or more, more preferably 20 or more, better still 30 or more. The greater N is and, as a result, the greater the total number of layers of the powder fractions formed, the more homogeneous the powder mixture becomes in the sense of uniformity of content.

The number m may represent 0 or 1 within the scope of the process according to the invention. If m denotes 0 the last fraction added to the mixing apparatus, preferably screened into it, in a layer is the last portion of the micronised compound of formula 1. If m represents the number 1, the last fraction added to the mixing apparatus, preferably screened into it, in a layer is the last portion of the physiologically acceptable excipient. This may prove advantageous inasmuch as, when m=1, any residues of the last fraction of the active substance still remaining in the screening unit can be carried into the mixing unit by means of the last portion of excipient.

Preferably, the first portion of the N+m portions of the excipient is put in first, and then the first portion of the N portions of the active substance is placed in the mixing container. Whereas within the scope of the process according to the invention the individual components are normally added in roughly equal portions, it may be advantageous in some cases if the first of the N+m portions of excipient which is put into the mixing apparatus has a larger volume than the subsequent portions of excipient.

The inhalable powders according to the invention may also be prepared by first of all producing a mixture of active substance and excipient according to the method described above and then mixing the mixture thus obtained with more excipient. This may be done using the method described above, by mixing N batches of the active substance/excipient mixture layer by layer with N+m batches of other excipient.

The excipient used in the inhalable powders according to the invention preferably has an average particle size of 17-120 μm, preferably about 17-90 μm, particularly preferably about 20-60 μm. The excipient may optionally also be a mixture of coarser excipient with an average particle size of 17 to 75 μm and finer excipient with an average particle size of 1 to 9 μm, wherein the proportion of finer excipient in the total quantity of excipient may be 1 to 20%. If the inhalable powders which may be produced using the process according to the invention contain a mixture of coarser and finer excipient fractions, it is preferable according to the invention to prepare inhalable powders wherein the coarser excipient has an average particle size of 17 to 50 μm, most preferably 20 to 30 μm, and the finer excipient has an average particle size of 2 to 8 μm, most preferably 3 to 7 μm. By average particle size is meant here the 50% value of the volume distribution measured with a laser diffractometer using the dry dispersion method. For the measurement of the mean particle size by this method see again the disclosure of WO 03/078429 (page 21 ff).

In the case of an excipient mixture of coarser and finer excipient fractions, the preferred processes according to the invention are those that produce inhalable powders in which the proportion of finer excipient constitutes 3 to 15%, most preferably 5 to 10% of the total amount of excipient.

The percentages given within the scope of the present invention are always percent by weight.

If the excipient used is one of the abovementioned mixtures of coarser excipient and finer excipient, it is again expedient according to the invention to produce the excipient mixture using the process according to the invention from N roughly equal portions of the finer excipient fraction with N+m roughly equal portions of the coarser excipient fraction. In such a case it is advisable first to generate the above-mentioned excipient mixture from the above-mentioned excipient fractions, and then to produce from it the total mixture including the active substance using the process according to the invention. For example, the excipient mixture may be obtained as follows, using the process according to the invention. The two components are preferably added through a screening granulator with a mesh size of 0.1 to 2 mm, most preferably 0.3 to 1 mm, even more preferably 0.3 to 0.6 mm. Preferably the first fraction of the N+m portions of the coarser excipient is put in first and then the first portion of the N portions of the finer excipient fraction is added to the mixing container. The two components are added alternately by screening them in layer by layer.

After the preparation of the excipient mixture, the inhalable powder is produced from the mixture and the desired active substance using the process according to the invention. The two components are preferably added through a screening granulator with a mesh size of 0.1 to 2 mm, most preferably 0.3 to 1 mm, even more preferably 0.3 to 0.6 mm.

Preferably, the first portion of the N+m portions of the excipient mixture is put in and then the first portion of the N portions of the active substance is added to the mixing container. The two components are preferably added through a screening unit in alternate layers, in more than 20, preferably more than 25, most preferably more than 30 layers. For example, with a desired total amount of powder of 30-35 kg containing 0.3-0.5% of active substance, for example, and using common excipients, the two components can be screened in in about 30 to 60 layers each (N=30-60). As will be clearly apparent to anyone skilled in the art, the process can equally well be carried out with N>60 to achieve the desired effect of the maximum possible homogeneity of the powder mixture.

### Indications

The inhalable powders according to the invention are characterised by their multiplicity of possible applications in the therapeutic field. Particular mention should be made according to the invention of those applications for which the compounds of formula 1 according to the invention are preferably used on the basis of their pharmaceutical activity as betamimetics.

Accordingly, in another aspect, the present invention relates to the above-mentioned inhalable powders as pharmaceutical compositions. The present invention also relates to the use of the above-mentioned inhalable powders for preparing a pharmaceutical composition for the treatment of respiratory complaints.

The present invention preferably relates to the use of the above-mentioned inhalable powders for preparing a pharmaceutical composition for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably, the inhalable powders according to the invention are used to prepare a pharmaceutical composition for the treatment of obstructive pulmonary diseases selected from the group consisting of COPD (chronic obstructive pulmonary disease), bronchial asthma, paediatric asthma, severe asthma, acute asthma attacks and chronic bronchitis, while their use for preparing a pharmaceutical composition for the treatment of bronchial asthma is particularly preferred according to the invention.

Also preferably, the inhalable powders according to the invention are used to prepare a pharmaceutical composition for the treatment of pulmonary emphysema which has its origins in COPD (chronic obstructive pulmonary disease) or a1-proteinase inhibitor deficiency.

Also preferably, the inhalable powders according to the invention are used to prepare a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

Also preferably, the inhalable powders according to the invention are used to prepare a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

Also preferably, the inhalable powders according to the invention are used to prepare a pharmaceutical composition for the treatment of cystic fibrosis or mucoviscidosis.

Also preferably, the inhalable powders according to the invention are used to prepare a pharmaceutical composition for the treatment of bronchitis, such as for example bronchitis caused by bacterial or viral infection, allergic bronchitis and toxic bronchitis.

Also preferably, the inhalable powders according to the invention are used to prepare a pharmaceutical composition for the treatment of bronchiectasis.

Also preferably, the inhalable powders according to the invention are used to prepare a pharmaceutical composition for the treatment of ARDS (adult respiratory distress syndrome).

Also preferably, the inhalable powders according to the invention are used to prepare a pharmaceutical composition for the treatment of pulmonary oedema, for example toxic pulmonary oedema after aspiration or inhalation of toxic substances and foreign substances.

Particularly preferably the present invention relates to the use of the inhalable powders according to the invention for preparing a pharmaceutical composition for the treatment of asthma or COPD. Also of particular importance is the above-mentioned use of the inhalable powders according to the invention for preparing a pharmaceutical composition for once-a-day treatment of inflammatory and obstructive respiratory complaints, particularly for the once-a-day treatment of asthma or COPD.

The present invention also relates to a process for the treatment of the above-mentioned diseases, characterised in that one or more of the above-mentioned inhalable powders according to the invention are administered in therapeutically effective amounts. The present invention preferably also relates to processes for the treatment of asthma or COPD, characterised in that one or more of the above-mentioned inhalable powders according to the invention are administered once a day in therapeutically effective amounts.

The examples of synthesis described below serve to illustrate the invention in more detail. However, they are intended only as examples of procedures to illustrate the invention without restricting it to the subject matter described in an exemplifying capacity hereinafter.

### Preparation of the Compounds of Formula 1

### Example 1

### N-(5-{2-[1,1-dimethyl-3-(4-methyl-2-oxo-4H-benzo[d][1,3]oxazin-1-yl)-propylamino]-1-hydroxy-ethyl}-2-hydroxy-phenyl)-methanesulphonamide

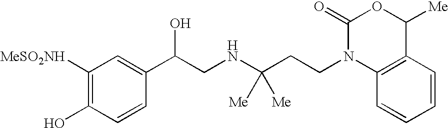

The compound is known from EP 43940. The individual diastereomers of this embodiment may be obtained by common methods known in the art.

### Example 2

### N-(5-{2-[1,1-dimethyl-3-(2-oxo-4H-benzo[d][1,3]oxazin-1-yl)-propylamino]-1-hydroxy-ethyl}-2-hydroxy-phenyl)-methanesulphonamide

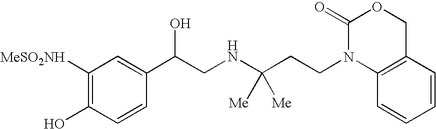

The compound is known from EP 43940. The (R)- and (S)-enantiomers of this embodiment may be obtained by common methods known in the art.

### Example 3

### N-(5-{2-[3-(4-ethyl-2-oxo-4H-benzo[d][1,3]oxazin-1-yl)-1,1-dimethyl-propylamino]-1-hydroxy-ethyl}-2-hydroxy-phenyl)-methanesulphonamide

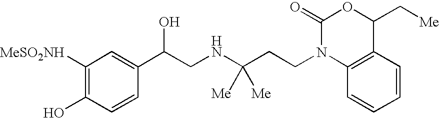

The compound is known from EP 43940. The individual diastereomers of this embodiment may be obtained by common methods known in the art.

### Example 4

### N-(5-{2-[3-(4,4-dimethyl-2-oxo-4H-benzo[d][1,3]oxazin-1-yl)-1,1-dimethyl-propylamino]-1-hydroxy-ethyl}-2-hydroxy-phenyl)-methanesulphonamide

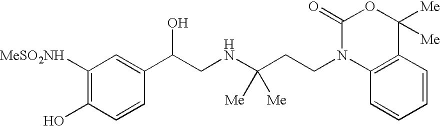

The compound is known from EP 43940. The (R)- and (S)-enantiomers of this embodiment may be obtained by common methods known in the art.

### Example 5

### N-(2-hydroxy-5-{1-hydroxy-2-[3-(6-hydroxy-4,4-dimethyl-2-oxo-4H-benzo[d][1,3]oxazin-1-yl)-1,1-dimethyl-propylamino]-ethyl}-phenyl)-methanesulphonamide

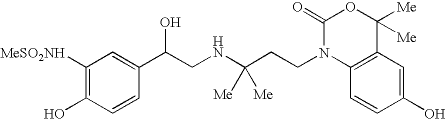

The compound is known from EP 43940. The (R)- and (S)-enantiomers of this embodiment may be obtained by common methods known in the art.

### Example 6

### N-(2-hydroxy-5-{1-hydroxy-2-[3-(6-methoxy-4,4-dimethyl-2-oxo-4H-benzo[d][1,3]oxazin-1-yl)-1,1-dimethyl-propylamino]-ethyl}-phenyl)-methanesulphonamide

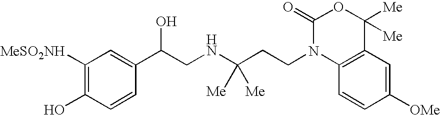

The compound is known from EP 43940. The (R)- and (S)-enantiomers of this embodiment may be obtained by common methods known in the art.

The examples of synthesis described below serve to illustrate new compounds according to the invention in more detail. However, they are intended only as examples of procedures to illustrate the invention without restricting it to the subject matter described in an exemplifying capacity hereinafter.

HPLC method (method A): Symmetry C18 (Waters): 3.5 μm; 4.6×150 mm; column temperature: 20° C.; gradient: acetonitrile/phosphate buffer (pH 7) 20:80→80:20 in 30 minutes; flow: 1.0 mL/min; detection at 220 and 254 nm.

### Synthesis of Intermediate Products 1-8

### Intermediate product 1:1-(3-amino-3-methyl-butyl)-4,4-dipropyl-1,4-dihydro-benzo[d][1,3]oxazin-2-one

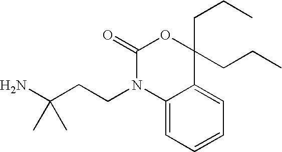

a) 4-(2-amino-phenyl)-heptan-4-ol: 90 mL (180.0 mmol) propylmagnesium chloride (2 M in ether) are added dropwise to a solution of 7.00 mL (54.0 mmol) methyl anthranilate in abs. THF (70 mL) at 0° C. within 30 minutes. The mixture is stirred for one hour at ambient temperature and then combined with 100 mL of 3 molar aqueous ammonium chloride solution and ethyl acetate. The phases are separated and the aqueous phase is exhaustively extracted with ethyl acetate. The combined organic phases are washed with potassium hydrogen carbonate solution and saturated sodium chloride solution and dried on sodium sulphate. The crude product is used in the next reaction step without further purification. Yield: 6.70 g (60%).

b) tert-butyl{3-[2-(1-hydroxy-1-propyl-butyl)-phenylamino]-1,1-dimethyl-propyl}-carbamate: 1.40 g (22.27 mmol) sodium cyanoborohydride are added to a solution of 3.10 g (14.05 mmol) 4-(2-amino-phenyl)-heptan-4-ol and 3.60 g (17.88 mmol) tert-butyl (1,1-dimethyl-3-oxo-propyl)-carbamate in methanol (40 mL) and acetic acid (6 mL). The mixture is stirred for 16 hours at ambient temperature, diluted with ethyl acetate, washed with 0.5 molar potassium hydrogen sulphate solution and saturated sodium chloride solution, dried on sodium sulphate and evaporated down in vacuo. The crude product is used in the next reaction step without further purification. Yield: 6.00 g (quantitative yield).

c) tert-butyl[1,1-dimethyl-3-(2-oxo-4,4-dipropyl-4H-benzo[d][1,3]oxazin-1-yl)-propyl]-carbamate: 8.85 mL (16.81 mmol) phosgene solution (20 wt. % in toluene) are slowly added dropwise at 0° C. to a solution of 6.00 g (15.28 mmol) tert-butyl{3-[2-(1-hydroxy-1-propyl-butyl)-phenylamino]-1,1-dimethyl-propyl}-carbamate and 5.32 mL (38.21 mmol) triethylamine in abs. THF (80 mL). The mixture is stirred for 2 hours at ambient temperature, diluted with ethyl acetate, combined with ice and made basic with saturated aqueous ammonia solution. The aqueous phase is exhaustively extracted with ethyl acetate and the combined organic phases are washed with saturated sodium chloride solution, dried on sodium sulphate and evaporated down in vacuo. After column chromatography (silica gel, cyclohexane/ethyl acetate=6:1) the product is obtained. Yield: 4.57 g (71%).

d) 1-(3-amino-3-methyl-butyl)-4,4-dipropyl-1,4-dihydro-benzo[d][1,3]oxazin-2-one: A solution of 4.20 g (10.03 mmol) tert-butyl[1,1-dimethyl-3-(2-oxo-4,4-dipropyl-4H-benzo[d][1,3]oxazin-1-yl)-propyl]-carbamate in 35 mL formic acid is stirred for 24 hours at ambient temperature and then poured onto ice. The aqueous phase is made basic with saturated aqueous ammonia solution and exhaustively extracted with ethyl acetate. The combined organic extracts are washed with sodium chloride solution, dried on sodium sulphate and evaporated down in vacuo. The residue is taken up in ethyl acetate (50 mL) and combined with 4 mL hydrochloric acid in ethyl acetate (saturated). The solution is evaporated down and twice mixed with a little ethanol and evaporated down in vacuo. Trituration of the residue with diisopropylether yields the product as the hygroscopic hydrochloride salt.

Yield: 2.60 g (73%).

### Intermediate product 2: 1-(3-amino-3-methyl-butyl)-4,4-diethyl-7-fluoro-1,4-dihydro-benzo[d][1,3]oxazin-2-one

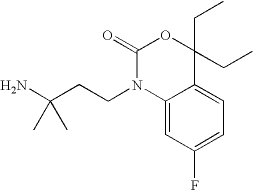

a) 3-(2-amino-4-fluoro-phenyl)-pentan-3-ol: The product is obtained analogously to intermediate product 1a by reacting methyl 2-amino-4-fluoro-benzoate and ethylmagnesium bromide in dichloromethane at −78° C. with heating to ambient temperature. Yield: 4.1 g (99%).

b) tert-butyl{3-[2-(1-ethyl-1-hydroxy-propyl)-5-fluoro-phenylamino]-1,1-dimethyl-propyl}-carbamate: The product is obtained analogously to intermediate product 1b starting from 3-(2-amino-4-fluoro-phenyl)-pentan-3-ol and tert-butyl (1,1-dimethyl-3-oxo-propyl)-carbamate. The crude product is purified by column chromatography (silica gel, dichloromethane/methanol=100:0→98:2). Yield: 7.70 g (99%).

c) tert-butyl[3-(4,4-diethyl-7-fluoro-2-oxo-4H-benzo[d][1,3]oxazin-1-yl)-1,1-dimethyl-propyl]-carbamate: The product is obtained analogously to intermediate product 1c starting from tert-butyl{3-[2-(1-ethyl-1-hydroxy-propyl)-5-fluoro-phenylamino]-1,1-dimethyl-propyl}-carbamate. Yield: 4.20 g (51%).

d) 1-(3-amino-3-methyl-butyl)-4,4-diethyl-7-fluoro-1,4-dihydro-benzo[d][1,3]oxazin-2-one: The product is prepared analogously to intermediate product 1d starting from tert-butyl[3-(4,4-diethyl-7-fluoro-2-oxo-4H-benzo[d][1,3]oxazin-1-yl)-1,1-dimethyl-propyl]-carbamate as the free base.

Yield: 2.90 g (96%); ESI-MS: [M+H]⁺=309.

### Intermediate product 3: 1-(3-amino-3-methyl-butyl)-spiro(cyclopropyl-1,4′-2H-3′,1′-benzoxazin)-2′-one

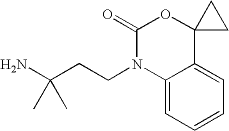

a) 1-(2-dibenzylamino-phenyl)-cyclopropanol: 2.45 mL (8.4 mmol) titanium tetraisopropoxide are slowly added dropwise at ambient temperature to a solution of 18.5 g (55.8 mmol) methyl 2-dibenzylamino-benzoate in 150 mL THF. After one hour's stirring 40.9 mL (122.7 mmol) ethylmagnesium bromide (3 M in diethyl ether) are added. The mixture is stirred for one hour, another 4 mL of 3 molar ethylmagnesium bromide solution are added and the mixture is stirred for 2 hours. The reaction mixture is combined with saturated ammonium chloride solution and extracted with ethyl acetate. The aqueous phase is combined with 1 molar hydrochloric acid until a clear solution is obtained and extracted with ethyl acetate. The combined organic phases are washed with sodium hydrogen carbonate solution and sodium chloride solution, dried on sodium sulphate and evaporated down. The residue is purified by chromatography (hexane/ethyl acetate=20:1). Yield: 10.0 g (54%).

b) 1-(2-amino-phenyl)-cyclopropanol: 9.90 g (30.1 mmol) 1-(2-dibenzylamino-phenyl)-cyclopropanol are dissolved in 70 mL methanol and hydrogenated in the presence of 1 g palladium on charcoal (10%) at 3 bar hydrogen pressure. The catalyst is removed by suction filtering, the filtrate is evaporated down and the residue is purified by chromatography (silica gel; cyclohexane/ethyl acetate=5:1). Yield: 1.80 g (40%).

c) tert-butyl{3-[2-(1-hydroxy-cyclopropyl)-phenylamino]-1,1-dimethyl-propyl}-carbamate: Prepared analogously to the method described for intermediate product 1b from 1.77 g (11.86 mmol) 1-(2-amino-phenyl)-cyclopropanol and 3.15 g (15.66 mmol) tert-butyl (1,1-dimethyl-3-oxo-propyl)-carbamate. The crude product obtained is purified by column chromatography (silica gel, cyclohexane/ethyl acetate 4:1). Yield: 2.60 g.

d) tert-butyl{1,1-dimethyl-3-[spiro(cycloproyl-1,4′-2H-3′,1′-benzoxazin)-2′-oxo-1-yl]-propyl}-carbamate: The product is obtained analogously to intermediate product 1c starting from 2.60 g (7.74 mmol) tert-butyl{3-[2-(1-hydroxy-cyclopropyl)-phenylamino]-1,1-dimethyl-propyl}-carbamate. A difference here is that there is no purification by column chromatography. Yield: 2.60 g.

e) 1-(3-amino-3-methyl-butyl)-spiro(cyclopropyl-1,4′-2H-3′,1′-benzoxazin)-2′-one: Obtained analogously to the method described for Intermediate 1d by reacting 3.10 g (8.60 mmol) tert-butyl{1,1-dimethyl-3-[spiro(cycloproyl-1,4′-2H-3′,1′-benzoxazin)-2′-oxo-1-yl]-propyl}-carbamate and 30 mL formic acid. Yield: 2.10 g (94%).

### Intermediate product 4: 1-(3-amino-3-methyl-butyl)-4,4-diethyl-1,4-dihydro-benzo[d][1,3]oxazin-2-one

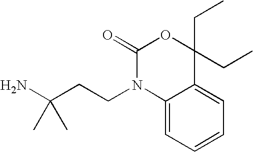

a) 3-(2-amino-phenyl)-pentan-3-ol: 100 mL of a 3 molar ethylmagnesium bromide solution in diethyl ether are added dropwise at −40° C. to a solution of 7.77 mL (60 mmol) 2-amino-methylbenzoic acid in 130 mL THF. The mixture is stirred overnight with heating to ambient temperature, combined with saturated ammonium chloride solution, acidified with 1 molar hydrochloric acid and extracted with ethyl acetate. The combined organic phases are extracted with water, dried on sodium sulphate and evaporated down. The crude product is further reacted directly. Yield: 10.9 g; mass spectroscopy: [M+H]⁺=180.

b) tert-butyl{3-[2-(1-ethyl-1-hydroxy-propyl)-phenylamino]-1,1-dimethyl-propyl}-carbamate: 3.16 g (47.7 mmol) sodium cyanoborohydride are added at ambient temperature to 5.70 g (31.8 mmol) 3-(2-amino-phenyl)-pentan-3-ol and 2.63 mL (47.7 mmol) acetic acid in 18 mL methanol. Then a solution of 7.04 g (35 mmol) tert-butyl (1,1-dimethyl-3-oxo-propyl)-carbamate in 18 mL methanol is slowly added dropwise. After the addition has ended the mixture is stirred for four hours, combined with 1 molar hydrochloric acid (development of gas) and then made basic with aqueous ammonia solution. It is extracted with ethyl acetate and the combined organic phases are washed with sodium chloride solution, dried on sodium sulphate and freed from the solvent. The residue is purified by column chromatography (silica gel, dichloromethane/methanol gradient with 0.1% ammonia). Yield: 4.25 g (37%); mass spectroscopy: [M+H]⁺=365.

c) tert-butyl[3-(4,4-diethyl-2-oxo-4H-benzo[d][1,3]oxazin-1-yl)-1,1-dimethyl-propyl]-carbamate: 2.91 g (9.6 mmol) triphosgene are added at 0 to 5° C. to a solution of 3.50 g (9.6 mmol) tert-butyl{3-[2-(1-ethyl-1-hydroxy-propyl)-phenylamino]-1,1-dimethyl-propyl}-carbamate and 3.37 mL (24 mmol) triethylamine in 35 mL THF. The mixture is left overnight at ambient temperature with stirring and the precipitate formed is suction filtered. The filtrate is evaporated down and the crude product remaining is further reacted directly.

Yield: 3.33 g; mass spectroscopy: [M+H]⁺=391.

d) 1-(3-amino-3-methyl-butyl)-4,4-diethyl-1,4-dihydro-benzo[d][1,3]oxazin-2-one: 25 mL trifluoroacetic acid are added dropwise, while being cooled with the ice bath, to a solution of 3.20 g tert-butyl[3-(4,4-diethyl-2-oxo-4H-benzo[d][1,3]oxazin-1-yl)-1,1-dimethyl-propyl]-carbamate (approx. 75%) in 25 mL dichloromethane. The mixture is stirred for 2 hours at ambient temperature, the solvents are distilled off and the acid residues are eliminated by repeated codistillation with toluene. To liberate the free base the residue is combined with 1 molar sodium hydroxide solution and extracted with ethyl acetate. The organic phases are dried on sodium sulphate and evaporated down. The free base is dissolved in 8 mL methanol and combined with ethereal hydrochloric acid. It is stirred overnight and the precipitate formed is suction filtered and washed with diethyl ether. Yield: 2.15 g (hydrochloride); mass spectroscopy: [M+H]⁺=291.

### Intermediate product 5: 1-(3-amino-3-methyl-butyl)-spiro(cyclohexane-1,4′-2H-3′,1′-benzoxazin)-2′-one

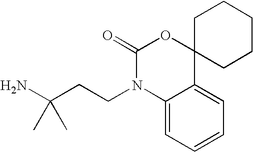

a) 1-(2-nitro-phenyl)cyclohexanol: 40.16 mL (80.32 mmol) phenylmagnesium chloride (2 M in THF) are added dropwise at −50° C. under nitrogen to a solution of 20.0 g (80.32 mmol) 2-nitro-iodobenzene in 150 mL THF. After 15 minutes stirring 9.98 mL (96.30 mmol) cyclohexanone are quickly added. The reaction mixture is heated to ambient temperature, stirred for two hours and combined with ammonium chloride solution. The aqueous phase is separated off and exhaustively extracted with ethyl acetate. The combined organic phases are washed with sodium chloride solution, dried on sodium sulphate and evaporated down. Column chromatography (silica gel, hexane/ethyl acetate=20:1) yields the product. Yield: 5.20 g (29%); R_{f}=0.26 (silica gel, hexane/ethyl acetate=10:1); ESI-MS: [M+H—H₂O]⁺=204.

b) 1-(2-amino-phenyl)-cyclohexanol: 5.20 g (16.45 mmol) 1-(2-nitro-phenyl)-cyclohexanol in 70 mL ethanol are hydrogenated for 4 hours in the presence of Raney nickel at ambient temperature and 3 bar hydrogen pressure. The catalyst is filtered off through Celite and the filtrate is evaporated down in vacuo. The residue is precipitated from hexane. Yield: 1.53 g (49%); R_{f}=0.38 (silica gel, hexane/ethyl acetate=4:1); ESI-MS: [M+H—H₂O]⁺=174.

c) tert-butyl{3-[2-(1-hydroxy-cyclohexyl)-phenylamino]-1,1-dimethyl-propyl}-carbamate: The compound is obtained analogously to intermediate product 1b from 1-(2-amino-phenyl)cyclohexanol and tert-butyl (1,1-dimethyl-3-oxo-propyl)-carbamate. Column chromatography (silica gel, hexane/ethyl acetate=7:1) yields the product. Yield: 2.65 g (66%); R_{f}=0.50 (silica gel, hexane/ethyl acetate=4:1).

d) tert-butyl{1,1-dimethyl-3-[spiro(cyclohexane-1,4′-2H-3′,1′-benzoxazin)-2′-oxo-1-yl]-propyl}-carbamate: Prepared analogously to intermediate product 1c from tert-butyl {3-[2-(1-hydroxy-cyclohexyl)-phenylamino]-1,1-dimethyl-propyl}-carbamate. Yield: 2.60 g (92%); R_{f}=0.38 (silica gel, hexane/ethyl acetate 4:1).

e) 1-(3-amino-3-methyl-butyl)-spiro(cyclohexane-1,4′-2H-3′,1′-benzoxazin)-2′-one: Prepared analogously to intermediate product 1d from tert-butyl[1,1-dimethyl-3-(spiro(cyclohexane-1,4′-2H-3′,1′-benzoxazin)-2′-oxo-1-yl)-propyl]-carbamate. Yield: 1.80 g (92%); R_{f}=0.10 (silica gel, dichloromethane/methanol/ammonia=95:5:0.5); ESI-MS: [M+H]⁺=303.

### Intermediate product 6: 1-(3-amino-3-methyl-butyl)-4,4-diethyl-8-methoxy-1,4-dihydro-benzo[d][1,3]oxazin-2-one

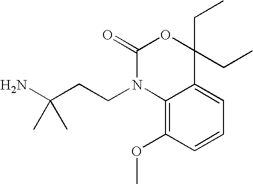

a) 3-(2-amino-3-methoxy-phenyl)-pentan-3-ol: The product is obtained analogously to intermediate product 1a by reacting methyl 2-amino-3-methoxy-benzoate and ethylmagnesium bromide in dichloromethane at −78° C.→RT. Yield: 5.20 g (92%); HPLC-MS: Rₜ=12.85 min. (method A); ESI-MS: [M+H]⁺=210.

b) tert-butyl{3-[2-(1-ethyl-1-hydroxy-propyl)-6-methoxy-phenylamino]-1,1-dimethyl-propyl}-carbamate: The product is obtained analogously to intermediate product 1b starting from 3-(2-amino-3-methoxy-phenyl)-pentan-3-ol and tert-butyl (1,1-dimethyl-3-oxo-propyl)-carbamate. The crude product is purified by column chromatography (silica gel, cyclohexane/ethyl acetate=4:1). Yield: 4.60 g (47%).

c) tert-butyl[3-(4,4-diethyl-8-methoxy-2-oxo-4H-benzo[d][1,3]oxazin-1-yl)-1,1-dimethyl-propyl]-carbamate: The product is obtained analogously to intermediate product 1c starting from tert-butyl{3-[2-(1-ethyl-1-hydroxy-propyl)-6-methoxy-phenylamino]-1,1-dimethyl-propyl}-carbamate. Yield: 4.60 g (94%).

d) 1-(3-amino-3-methyl-butyl)-4,4-diethyl-8-methoxy-1,4-dihydro-benzo[d][1,3]oxazin-2-one: The product is obtained analogously to intermediate product 1d starting from tert-butyl[3-(4,4-diethyl-8-methoxy-2-oxo-4H-benzo[d][1,3]oxazin-1-yl)-1,1-dimethyl-propyl]-carbamate as a free base. Yield: 3.00 g (93%); ESI-MS: [M+H]⁺=321.

### Intermediate product 7: 1-(3-amino-3-methyl-butyl)-4,4-diethyl-6-fluoro-1,4-dihydro-benzo[d][1,3]oxazin-2-one

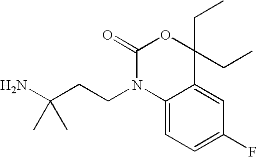

a) 3-(2-amino-5-fluoro-phenyl)-pentan-3-ol: Prepared analogously to intermediate product 1a from methyl 2-amino-5-fluoro-benzoate and ethylmagnesium bromide. The product obtained is purified by chromatography (silica gel, cyclohexane/ethyl acetate=8:1). Yield: 6.00 g (74%).

b) tert-butyl{3-[2-(1-ethyl-1-hydroxy-propyl)-4-fluoro-phenylamino]-1,1-dimethyl-propyl}-carbamate: The product is obtained analogously to intermediate product 1b starting from 3-(2-amino-5-fluoro-phenyl)-pentan-3-ol and tert-butyl (1,1-dimethyl-3-oxo-propyl)-carbamate. The crude product is purified by column chromatography (silica gel, hexane/ethyl acetate=6:1→2:1). Yield: 4.50 g (41%).

c) tert-butyl[3-(4,4-diethyl-6-fluoro-2-oxo-4H-benzo[d][1,3]oxazin-1-yl)-1,1-dimethyl-propyl]-carbamate: Prepared analogously to intermediate product 1c from tert-butyl{3-[2-(1-ethyl-1-hydroxy-propyl)-4-fluoro-phenylamino]-1,1-dimethyl-propyl}-carbamate. A difference here is that there is no purification by column chromatography. Yield: 4.8 g.

d) 1-(3-amino-3-methyl-butyl)-4,4-diethyl-6-fluoro-1,4-dihydro-benzo[d][1,3]oxazin-2-one: The target compound is prepared as a free base analogously to intermediate product 1d from tert-butyl[3-(4,4-diethyl-6-fluoro-2-oxo-4H-benzo[d][1,3]oxazin-1-yl)-1,1-dimethyl-propyl]-carbamate. Yield: 3.00 g (99%).

### Intermediate product 8: 1-(3-amino-3-methyl-butyl)-4,4-diethyl-6-methoxy-1,4-dihydro-benzo[d][1,3]oxazin-2-one

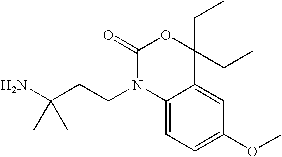

a) 3-(2-amino-5-methoxy-phenyl)-pentan-3-ol: the product is obtained by reacting 4.00 g (22 mmol) methyl 2-amino-5-methoxy-benzoate with 5 equivalents ethylmagnesium bromide in dichloromethane at −78° C.->RT. Yield: 4.47 g (97%).

b) tert-butyl{3-[2-(1-ethyl-1-hydroxy-propyl)-4-methoxy-phenylamino]-1,1-dimethyl-propyl}-carbamate: Prepared analogously to intermediate product 1b from 4.45 g (21 mmol) 3-(2-amino-5-methoxy-phenyl)-pentan-3-ol and 5.66 g (28 mmol) tert-butyl (1,1-dimethyl-3-oxo-propyl)-carbamate. Yield: 6.00 g (72%).

c) tert-butyl[3-(4,4-diethyl-6-methoxy-2-oxo-4H-benzo[d][1,3]oxazin-1-yl)-1,1-dimethyl-propyl]-carbamate: The product is prepared analogously to intermediate product 1c from 6.00 g (15.2 mmol) tert-butyl{3-[2-(1-ethyl-1-hydroxy-propyl)-4-methoxy-phenylamino]-1,1-dimethyl-propyl}-carbamate. Yield: 3.10 g (48%).

d) 1-(3-amino-3-methyl-butyl)-4,4-diethyl-6-methoxy-1,4-dihydro-benzo[d][1,3]oxazin-2-one: Prepared analogously to intermediate product 1d from 3.10 g (8.5 mmol) tert-butyl[3-(4,4-diethyl-6-methoxy-2-oxo-4H-benzo[d][1,3]oxazin-1-yl)-1,1-dimethyl-propyl]-carbamate. The product is isolated as the free base and not converted into a hydrochloride salt. Yield: 2.20 g (98%).

### Example 7

### N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide

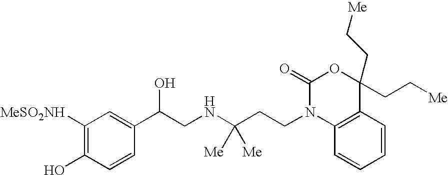

a) N-(2-benzyloxy-5-{2-[1,1-dimethyl-3-(2-oxo-4,4-dipropyl-4H-benzo[d][1,3]oxazin-1-yl)-propylamino]-1-hydroxy-ethyl}-phenyl)-methanesulphonamide: 86 μl (0.619 mmol) triethylamine are added at ambient temperature under a nitrogen atmosphere to a solution of 200 mg (0.564 mmol) 1-(3-amino-3-methyl-butyl)-4,4-dipropyl-1,4-dihydro-benzo[d][1,3]oxazin-2-one hydrochloride in 5 mL THF. The mixture is stirred for 30 minutes, 218 mg (0.575 mmol) N-[2-benzyloxy-5-(2-ethoxy-2-hydroxy-acetyl)-phenyl]-methanesulphonamide are added and the mixture is stirred for a further 2 hours at ambient temperature. The mixture is cooled to 10° C., combined with 51 mg (2.34 mmol) lithium borohydride, heated to ambient temperature and stirred for one hour. It is cooled to 10° C. again and diluted with 15 mL water and 20 mL dichloromethane. The aqueous phase is separated off and extracted with dichloromethane. The combined organic phases are dried on sodium sulphate and evaporated down in vacuo. The residue is dissolved in 8 mL ethyl acetate and acidified to pH 2 by the addition of saturated hydrochloric acid in ethyl acetate. The precipitate formed is filtered off, washed with ethyl acetate and evaporated down. Yield: 260 mg (67%, hydrochloride), HPLC: Rₜ=19.8 minutes (method A).

b) N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide: 260 mg (0.386 mmol) N-(2-benzyloxy-5-{2-[1,1-dimethyl-3-(2-oxo-4,4-dipropyl-4H-benzo[d][1,3]oxazin-1-yl)-propylamino]-1-hydroxy-ethyl}-phenyl)-methanesulphonamide hydrochloride in 8 mL methanol are hydrogenated in the presence of 26 mg palladium on charcoal (10%) at ambient temperature. The catalyst is filtered off through Celite and washed with methanol. The filtrate is evaporated down in vacuo and the residue is stirred into diethyl ether.

Yield: 120 mg (53%, hydrochloride); mass spectroscopy: [M+H]⁺=548; HPLC: Rₜ=14.7 minutes (method A).

The (R)- and (S)-enantiomers of this embodiment may be obtained by common methods known in the art. The (R)-enantiomer of this embodiment is of particular importance according to the invention.

### Example 8

### N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide

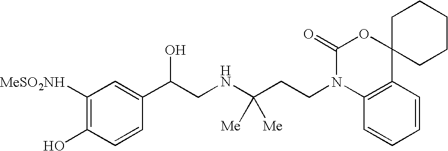

a) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide: Prepared analogously to the process described for Example 7a from 250 mg (0.66 mmol) N-[2-benzyloxy-5-(2-ethoxy-2-hydroxy-acetyl)-phenyl]-methanesulphonamide and 200 mg (0.66 mmol) 1-(3-amino-3-methyl-butyl)-spiro(cyclohexane-1,4′-2H-3′,1′-benzoxazin)-2′-one. A difference here is that the product obtained as the hydrochloride is also purified by chromatography (silica gel, dichloromethane/methanol=50:1).

Yield: 190 mg (46%), HPLC: Rₜ=17.8 minutes (method A).

b) N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide: 190 mg (0.31 mmol) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide are hydrogenated analogously to Example 7b. After separation of the catalyst the filtrate is freed from the solvent, combined with 8 mL ethyl acetate and acidified to pH 2 by the addition of hydrochloric acid in ethyl acetate. The solvent is distilled off and the residue is stirred in diethyl ether and filtered. Yield: 40 mg (23%, hydrochloride); mass spectroscopy: [M+H]⁺=532; HPLC: Rₜ=11.8 minutes (method A).

The (R)- and (S)-enantiomers of this embodiment may be obtained by common methods known in the art. Particular importance attaches to the (R)-enantiomer of this embodiment according to the invention.

### Example 9

### N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide

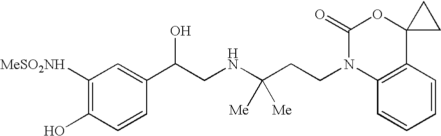

a) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide: 292 mg (0.77 mmol) N-[2-benzyloxy-5-(2-ethoxy-2-hydroxy-acetyl)-phenyl]-methanesulphonamide and 200 mg (0.77 mmol) 1-(3-amino-3-methyl-butyl)-spiro(cyclopropyl-1,4′-2H-3′,1′-benzoxazin)-2′-one are reacted and worked up analogously to Example 7a. The crude product is combined with 8 mL ethyl acetate and acidified to pH 2 with hydrochloric acid in ethyl acetate. The solvent is distilled off and the residue is stirred in diethyl ether. Yield: 400 mg (84%, hydrochloride), HPLC: Rₜ=15.2 minutes (method A).

b) N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide: the product is prepared analogously to Example 1b from 400 mg (0.65 mmol) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide hydrochloride. Yield: 230 mg (67%, hydrochloride); mass spectroscopy: [M+H]⁺=490; HPLC: Rₜ=8.9 minutes (method A).

The (R)- and (S)-enantiomers of this embodiment may be obtained by common methods known in the art. Particular importance attaches to the (R)-enantiomer of this embodiment according to the invention.

### Example 10

### N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide

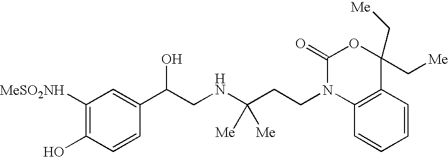

379 mg (1 mmol) N-[2-benzyloxy-5-(2-ethoxy-2-hydroxy-acetyl)-phenyl]-methanesulphonamide and 290 mg (1 mmol) 1-(3-amino-3-methyl-butyl)-4,4-diethyl-1,4-dihydro-benzo[d][1,3]oxazin-2-on are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for a further 30 minutes. The mixture is filtered through aluminium oxide (basic), washed repeatedly with dichloromethane/methanol (15:1) and evaporated down. The crude product thus obtained is purified by chromatography (dichloromethane with 0-10% methanol/ammonia=9:1). The benzylether thus obtained is dissolved in 10 mL methanol and hydrogenated with palladium on charcoal as catalyst at 1 bar hydrogen pressure. Then the catalyst is filtered off and the filtrate is evaporated down. Yield: 338 mg (65% over 2 steps); mass spectroscopy: [M+H]⁺=520.

The (R)- and (S)-enantiomers of this embodiment may be obtained by common methods known in the art. Particular importance attaches to the (R)-enantiomer of this embodiment according to the invention. The angle of rotation of (R)—N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide hydrochloride (cocrystallised with a molecule of acetone) is −28.8° (c=1%, in methanol at 20° C.).

### Example 11

### N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide

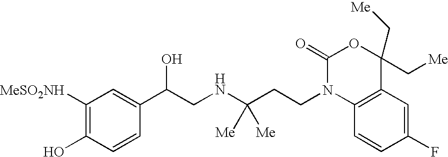

a) N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide: Reaction of 246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-6-fluoro-1,4-dihydro-benzo[D][1,3]oxazin-2-one analogously to Example 7a. One difference is that the preparation of the hydrochloride is omitted. Instead, the free base is purified by chromatography (reverse phase, acetonitrile/water gradient with 0.1% trifluoroacetic acid).

Yield: 180 mg (trifluoroacetate), HPLC: Rₜ=17.4 minutes (method A).

b) N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide: 175 mg of —N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide trifluoroacetate in 9 mL methanol are hydrogenated in the presence of 40 mg Raney nickel at ambient temperature and 3 bar hydrogen pressure. The catalyst is filtered off and the filtrate is freed from the solvent.

Yield: 131 mg (trifluoroacetate); mass spectroscopy: [M+H]⁺=538.

The (R)- and (S)-enantiomers of this embodiment may be obtained by common methods known in the art. Particular importance attaches to the (R)-enantiomer of this embodiment according to the invention.

### Example 12

### N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide

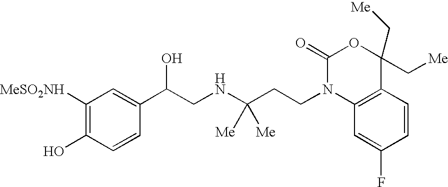

a) N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)-methanesulphonamide: 246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-7-fluoro-1,4-dihydro-benzo[d][1,3]oxazin-2-one are reacted and worked up analogously to Example 7a. A difference is that the production of the hydrochloride is omitted and the free base is purified by chromatography (reverse phase, acetonitrile/water gradient with 0.1% trifluoroacetic acid).

Yield: 220 mg (trifluoroacetate), HPLC: Rₜ=17.7 minutes (method A).

b) N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide: Prepared analogously to Example 11b from 210 mg of N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)methanesulphonamide trifluoroacetate.

Yield: 154 mg (trifluoroacetate); mass spectroscopy: [M+H]⁺=538.

The (R)- and (S)-enantiomers of this embodiment may be obtained by common methods known in the art. Particular importance attaches to the (R)-enantiomer of this embodiment according to the invention.

### Example 13

### N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide

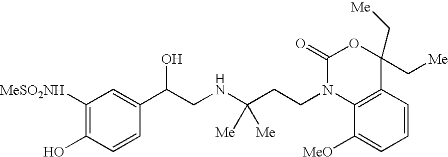

a) N-(2-benzyloxy-5-{2-[3-(4,4-diethyl-8-methoxy-2-oxo-4H-benzo[d][1,3]oxazin-1-yl)-1,1-dimethyl-propylamino]-1-hydroxy-ethyl}-phenyl)-methanesulphonamide: reaction of 237 mg (0.625 mmol) N-[2-benzyloxy-5-(2-ethoxy-2-hydroxy-acetyl)-phenyl]-methanesulphonamide and 200 mg (0.624 mmol) 1-(3-amino-3-methyl-butyl)-4,4-diethyl-8-methoxy-1,4-dihydro-benzo[d][1,3]oxazin-2-one analogously to Example 7a. The crude product is dissolved in ethyl acetate and acidified to pH 2 with hydrochloric acid in ethyl acetate. The solvent is distilled off and the residue is stirred in diethyl ether. Then the hydrochloride thus obtained (330 mg) is further purified by chromatography.

Yield: 90 mg (trifluoroacetate), HPLC: Rₜ=17.6 minutes (method A).

b) N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide: 80 mg (0.118 mmol) N-(2-benzyloxy-5-{2-[3-(4,4-diethyl-8-methoxy-2-oxo-4H-benzo[d][1,3]oxazin-1-yl)-1,1-dimethyl-propylamino]-1-hydroxy-ethyl}-phenyl)-methanesulphonamide trifluoroacetate are hydrogenated analogously to Example 11b. Yield: 70 mg (trifluoroacetate); mass spectroscopy: [M+H]⁺=550.

The (R)- and (S)-enantiomers of this embodiment may be obtained by common methods known in the art. Particular importance attaches to the (R)-enantiomer of this embodiment according to the invention.

### Example 14

### N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide

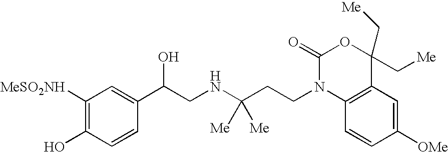

a) N-(2-benzyloxy-5-{2-[3-(4,4-diethyl-6-methoxy-2-oxo-4H-benzo[d][1,3]oxazin-1-yl)-1,1-dimethyl-propylamino]-1-hydroxy-ethyl}-phenyl)-methanesulphonamide: 235 mg (0.619 mmol) N-[2-benzyloxy-5-(2-ethoxy-2-hydroxy-acetyl)-phenyl]-methanesulphonamide and 200 mg (0.624 mmol) 1-(3-amino-3-methyl-butyl)-4,4-diethyl-6-methoxy-1,4-dihydro-benzo[d][1,3]oxazin-2-one are reacted analogously to Example 7a. One difference is that the crude product is not precipitated as the hydrochloride, but purified by chromatography (reverse phase, acetonitrile/water gradient with 0.1% trifluoroacetic acid).

Yield: 150 mg (trifluoroacetate), HPLC: Rₜ=16.9 minutes (method A).

b) N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide: The target compound is prepared from N-(2-benzyloxy-5-{2-[3-(4,4-diethyl-6-methoxy-2-oxo-4H-benzo[d][1,3]oxazin-1-yl)-1,1-dimethyl-propylamino]-1-hydroxy-ethyl}-phenyl)-methanesulphonamide trifluoroacetate analogously to Example 11b. Mass spectroscopy: [M+H]⁺=550.

The (R)- and (S)-enantiomers of this embodiment may be obtained by common methods known in the art. Particular importance attaches to the (R)-enantiomer of this embodiment according to the invention.

### Preparation of the Powder Formulations According to the Invention

The inhalable powders may be prepared for example using the following machines and equipment:
mixing container or powder mixer: Turbulamischer 2 L, type 2C; manufactured by Willy A. Bachofen AG, CH-4500 Basle
hand-held screen: 0.315 mm mesh size

The filling of the empty inhalation capsules with inhalable powders containing active substance may be done manually or by machine. The following apparatus may be used.

Capsule filling machine: MG2, type G100, manufacturer: MG2 S.r.l, I-40065 Pian di Macina di Pianoro (BO), Italy

Powder mixture: In order to prepare the powder mixture 99.5 g excipient (lactose monohydrate 200 mesh with an average particle size of 25-50 μm, which varies from one batch to another) and 0.5 g micronised compound of formula 1 are used. The proportion of active substance in the 100 g inhalable powder obtained is 0.5%.

The excipient is placed in a suitable mixing container through a hand-held screen with a mesh size of 0.315 mm. Then 0.5 g of micronised compound of formula 1 and 9.5 g of excipient are screened in in alternate layers. The excipient and the active substance are added in 7 and 6 layers, respectively (premix I). The constituents screened in are then mixed (mixing: 30 rpm/30 min).

10 g of premix 1 and 90 g excipient are then added in alternate layers through the same hand-held screen with a mesh size of 0.315 mm by screening into a suitable mixing container. The excipient and the premix I are added in 8-10 layers (final mix). The constituents screened in are then mixed (mixing: 30 rpm/30 min).

The following inhalable powders may be obtained according to or analogously to the procedure described hereinbefore:

| | | | |
|---|---|---|---|
| A) | active substance = Example 6 | 0.1 | g |
| | lactose monohydrate: | 99.9 | g |
| | Total: | 100.0 | g |
| B) | active substance = Example 6 | 0.2 | g |
| | lactose monohydrate: | 99.8 | g |
| | Total: | 100.0 | g |
| C) | active substance = Example 6 | 0.3 | g |
| | lactose monohydrate: | 99.7 | g |
| | Total: | 100.0 | g |
| D) | active substance = Example 6 | 0.4 | g |
| | lactose monohydrate: | 99.6 | g |
| | Total: | 100.0 | g |
| E) | active substance = Example 6 | 0.5 | g |
| | lactose monohydrate: | 99.5 | g |
| | Total: | 100.0 | g |
| F) | active substance = Example 7 | 0.1 | g |
| | lactose monohydrate: | 99.9 | g |
| | Total: | 100.0 | g |
| G) | active substance = Example 7 | 0.2 | g |
| | lactose monohydrate: | 99.8 | g |
| | Total: | 100.0 | g |
| H) | active substance = Example 7 | 0.3 | g |
| | lactose monohydrate: | 99.7 | g |
| | Total: | 100.0 | g |
| I) | active substance = Example 7 | 0.4 | g |
| | lactose monohydrate: | 99.6 | g |
| | Total: | 100.0 | g |
| J) | active substance = Example 7 | 0.5 | g |
| | lactose monohydrate: | 99.5 | g |
| | Total: | 100.0 | g |
| K) | active substance = Example 8 | 0.1 | g |
| | lactose monohydrate: | 99.9 | g |
| | Total: | 100.0 | g |
| L) | active substance = Example 8 | 0.2 | g |
| | lactose monohydrate: | 99.8 | g |
| | Total: | 100.0 | g |
| M) | active substance = Example 8 | 0.3 | g |
| | lactose monohydrate: | 99.7 | g |
| | Total: | 100.0 | g |
| N) | active substance = Example 8 | 0.4 | g |
| | lactose monohydrate: | 99.6 | g |
| | Total: | 100.0 | g |
| O) | active substance = Example 8 | 0.5 | g |
| | lactose monohydrate: | 99.5 | g |
| | Total: | 100.0 | g |
| P) | active substance = Example 9 | 0.1 | g |
| | lactose monohydrate: | 99.9 | g |
| | Total: | 100.0 | g |
| Q) | active substance = Example 9 | 0.2 | g |
| | lactose monohydrate: | 99.8 | g |
| | Total: | 100.0 | g |
| R) | active substance = Example 9 | 0.3 | g |
| | lactose monohydrate: | 99.7 | g |
| | Total: | 100.0 | g |
| S) | active substance = Example 9 | 0.4 | g |
| | lactose monohydrate: | 99.6 | g |
| | Total: | 100.0 | g |
| T) | active substance = Example 9 | 0.5 | g |
| | lactose monohydrate: | 99.5 | g |
| | Total: | 100.0 | g |
| U) | active substance = Example 10 | 0.1 | g |
| | lactose monohydrate: | 99.9 | g |
| | Total: | 100.0 | g |
| V) | active substance = Example 10 | 0.2 | g |
| | lactose monohydrate: | 99.8 | g |
| | Total: | 100.0 | g |
| W) | active substance = Example 10 | 0.3 | g |
| | lactose monohydrate: | 99.7 | g |
| | Total: | 100.0 | g |
| X) | active substance = Example 10 | 0.4 | g |
| | lactose monohydrate: | 99.6 | g |
| | Total: | 100.0 | g |
| Y) | active substance = Example 10 | 0.5 | g |
| | lactose monohydrate: | 99.5 | g |
| | Total: | 100.0 | g |
| Z) | active substance = Example 11 | 0.1 | g |
| | lactose monohydrate: | 99.9 | g |
| | Total: | 100.0 | g |
| AA) | active substance = Example 11 | 0.2 | g |
| | lactose monohydrate: | 99.8 | g |
| | Total: | 100.0 | g |
| BB) | active substance = Example 11 | 0.3 | g |
| | lactose monohydrate: | 99.7 | g |
| | Total: | 100.0 | g |
| CC) | active substance = Example 11 | 0.4 | g |
| | lactose monohydrate: | 99.6 | g |
| | Total: | 100.0 | g |
| DD) | active substance = Example 11 | 0.5 | g |
| | lactose monohydrate: | 99.5 | g |
| | Total: | 100.0 | g |
| EE) | active substance = Example 13 | 0.1 | g |
| | lactose monohydrate: | 99.9 | g |
| | Total: | 100.0 | g |
| FF) | active substance = Example 13 | 0.2 | g |
| | lactose monohydrate: | 99.8 | g |
| | Total: | 100.0 | g |
| GG) | active substance = Example 13 | 0.3 | g |
| | lactose monohydrate: | 99.7 | g |
| | Total: | 100.0 | g |
| HH) | active substance = Example 13 | 0.4 | g |
| | lactose monohydrate: | 99.6 | g |
| | Total: | 100.0 | g |
| II) | active substance = Example 13 | 0.5 | g |
| | lactose monohydrate: | 99.5 | g |
| | Total: | 100.0 | g |
| JJ) | active substance = Example 14 | 0.1 | g |
| | lactose monohydrate: | 99.9 | g |
| | Total: | 100.0 | g |
| KK) | active substance = Example 14 | 0.2 | g |
| | lactose monohydrate: | 99.8 | g |
| | Total: | 100.0 | g |
| LL) | active substance = Example 14 | 0.3 | g |
| | lactose monohydrate: | 99.7 | g |
| | Total: | 100.0 | g |
| MM) | active substance = Example 14 | 0.4 | g |
| | lactose monohydrate: | 99.6 | g |
| | Total: | 100.0 | g |
| NN) | active substance = Example 14 | 0.5 | g |
| | lactose monohydrate: | 99.5 | g |
| | Total: | 100.0 | g |
| OO) | active substance = Example 6 | 0.1 | g |
| | lactose anhydrate: | 99.9 | g |
| | Total: | 100.0 | g |
| PP) | active substance = Example 6 | 0.2 | g |
| | lactose anhydrate: | 99.8 | g |
| | Total: | 100.0 | g |
| QQ) | active substance = Example 6 | 0.3 | g |
| | lactose anhydrate: | 99.7 | g |
| | Total: | 100.0 | g |
| RR) | active substance = Example 6 | 0.4 | g |
| | lactose anhydrate: | 99.6 | g |
| | Total: | 100.0 | g |
| SS) | active substance = Example 6 | 0.5 | g |
| | lactose anhydrate: | 99.5 | g |
| | Total: | 100.0 | g |
| TT) | active substance = Example 7 | 0.1 | g |
| | lactose anhydrate: | 99.9 | g |
| | Total: | 100.0 | g |
| UU) | active substance = Example 7 | 0.2 | g |
| | lactose anhydrate: | 99.8 | g |
| | Total: | 100.0 | g |
| VV) | active substance = Example 7 | 0.3 | g |
| | lactose anhydrate: | 99.7 | g |
| | Total: | 100.0 | g |
| WW) | active substance = Example 7 | 0.4 | g |
| | lactose anhydrate: | 99.6 | g |
| | Total: | 100.0 | g |
| XX) | active substance = Example 7 | 0.5 | g |
| | lactose anhydrate: | 99.5 | g |
| | Total: | 100.0 | g |
| YY) | active substance = Example 8 | 0.1 | g |
| | lactose anhydrate: | 99.9 | g |
| | Total: | 100.0 | g |
| ZZ) | active substance = Example 8 | 0.2 | g |
| | lactose anhydrate: | 99.8 | g |
| | Total: | 100.0 | g |
| AAA) | active substance = Example 8 | 0.3 | g |
| | lactose anhydrate: | 99.7 | g |
| | Total: | 100.0 | g |
| BBB) | active substance = Example 8 | 0.4 | g |
| | lactose anhydrate: | 99.6 | g |
| | Total: | 100.0 | g |
| CCC) | active substance = Example 8 | 0.5 | g |
| | lactose anhydrate: | 99.5 | g |
| | Total: | 100.0 | g |
| DDD) | active substance = Example 9 | 0.1 | g |
| | lactose anhydrate: | 99.9 | g |
| | Total: | 100.0 | g |
| EEE) | active substance = Example 9 | 0.2 | g |
| | lactose anhydrate: | 99.8 | g |
| | Total: | 100.0 | g |
| FFF) | active substance = Example 9 | 0.3 | g |
| | lactose anhydrate: | 99.7 | g |
| | Total: | 100.0 | g |
| GGG) | active substance = Example 9 | 0.4 | g |
| | lactose anhydrate: | 99.6 | g |
| | Total: | 100.0 | g |
| HHH) | active substance = Example 9 | 0.5 | g |
| | lactose anhydrate: | 99.5 | g |
| | Total: | 100.0 | g |
| III) | active substance = Example 10 | 0.1 | g |
| | lactose anhydrate: | 99.9 | g |
| | Total: | 100.0 | g |
| JJJ) | active substance = Example 10 | 0.2 | g |
| | lactose anhydrate: | 99.8 | g |
| | Total: | 100.0 | g |
| KKK) | active substance = Example 10 | 0.3 | g |
| | lactose anhydrate: | 99.7 | g |
| | Total: | 100.0 | g |
| LLL) | active substance = Example 10 | 0.4 | g |
| | lactose anhydrate: | 99.6 | g |
| | Total: | 100.0 | g |
| MMM) | active substance = Example 10 | 0.5 | g |
| | lactose anhydrate: | 99.5 | g |
| | Total: | 100.0 | g |
| NNN) | active substance = Example 11 | 0.1 | g |
| | lactose anhydrate: | 99.9 | g |
| | Total: | 100.0 | g |
| OOO) | active substance = Example 11 | 0.2 | g |
| | lactose anhydrate: | 99.8 | g |
| | Total: | 100.0 | g |
| PPP) | active substance = Example 11 | 0.3 | g |
| | lactose anhydrate: | 99.7 | g |
| | Total: | 100.0 | g |
| QQQ) | active substance = Example 11 | 0.4 | g |
| | lactose anhydrate: | 99.6 | g |
| | Total: | 100.0 | g |
| RRR) | active substance = Example 11 | 0.5 | g |
| | lactose anhydrate: | 99.5 | g |
| | Total: | 100.0 | g |
| SSS) | active substance = Example 13 | 0.1 | g |
| | lactose anhydrate: | 99.9 | g |
| | Total: | 100.0 | g |
| TTT) | active substance = Example 13 | 0.2 | g |
| | lactose anhydrate: | 99.8 | g |
| | Total: | 100.0 | g |
| UUU) | active substance = Example 13 | 0.3 | g |
| | lactose anhydrate: | 99.7 | g |
| | Total: | 100.0 | g |
| VVV) | active substance = Example 13 | 0.4 | g |
| | lactose anhydrate: | 99.6 | g |
| | Total: | 100.0 | g |
| WWW) | active substance = Example 13 | 0.5 | g |
| | lactose anhydrate: | 99.5 | g |
| | Total: | 100.0 | g |
| XXX) | active substance = Example 14 | 0.1 | g |
| | lactose anhydrate: | 99.9 | g |
| | Total: | 100.0 | g |
| YYY) | active substance = Example 14 | 0.2 | g |
| | lactose anhydrate: | 99.8 | g |
| | Total: | 100.0 | g |
| ZZZ) | active substance = Example 14 | 0.3 | g |
| | lactose anhydrate: | 99.7 | g |
| | Total: | 100.0 | g |
| AAAA | active substance = Example 14 | 0.4 | g |
| | lactose anhydrate: | 99.6 | g |
| | Total: | 100.0 | g |
| BBBB | active substance = Example 14 | 0.5 | g |
| | lactose anhydrate: | 99.5 | g |
| | Total: | 100.0 | g |

1. Inhalable powders, containing one or more enantiomerically pure compounds of general formula 1 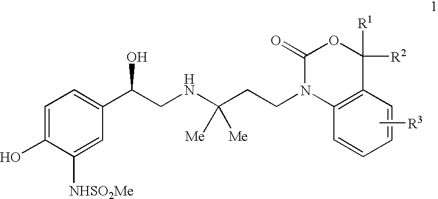
wherein
R¹ and R² independently of one another denote H, halogen or C₁₋₄-alkyl or together denote C₁₋₆-alkylene; and
R³ denotes H, halogen, OH, C₁₋₄-alkyl or O—C₁₋₄-alkyl;
optionally in the form of the pharmaceutically acceptable acid addition salts, hydrates or solvates thereof, optionally in admixture with one or more physiologically acceptable excipients.

2. Inhalable powders according to claim 1, containing one or more enantiomerically pure compounds of general formula 1, wherein
R¹ and R² which may be identical or different, denote hydrogen, fluorine, chlorine, methyl, ethyl, propyl, butyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, chlorine, OH, methyl, ethyl, methoxy, or ethoxy,
optionally in the form of the pharmaceutically acceptable acid addition salts, hydrates or solvates thereof, optionally in admixture with one or more physiologically acceptable excipients.

3. Inhalable powders according to claim 1, containing one or more enantiomerically pure compounds of general formula 1, wherein
R¹ and R² which may be identical or different, denote hydrogen, methyl, ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy,
optionally in the form of the pharmaceutically acceptable acid addition salts, hydrates or solvates thereof, optionally in admixture with one or more physiologically acceptable excipients.

4. Inhalable powders according to claim 1, containing one or more enantiomerically pure compounds of general formula 1, wherein
R¹ and R² which may be identical or different, ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy,
optionally in the form of the pharmaceutically acceptable acid addition salts, hydrates or solvates thereof, optionally in admixture with one or more physiologically acceptable excipients.

5. Inhalable powders according to claim 1, containing one or more enantiomerically pure compounds of general formula 1, wherein
R¹ and R² denote ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH or methoxy,
optionally in the form of the pharmaceutically acceptable acid addition salts, hydrates or solvates thereof, optionally in admixture with one or more physiologically acceptable excipients.

6. Inhalable powders according to claim 1, containing one or more enantiomerically pure compounds of general formula 1 in the form of their free bases.

7. Inhalable powders according to claim 1, containing one or more enantiomerically pure compounds of general formula 1 in the form of the pharmaceutically acceptable acid addition salts thereof.

8. Inhalable powders according to claim 1, containing one or more enantiomerically pure compounds of general formula 1 in the form of the acid addition salts thereof of general formula 1-HX, 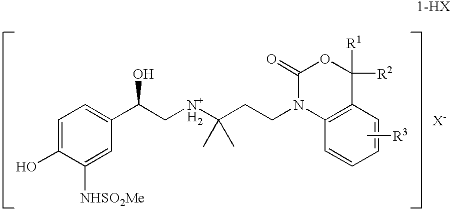
wherein
X⁻ denotes a mono- or polysubstituted negatively charged anion, preferably a mono- or polysubstituted negatively charged anion selected from among chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
and the groups R¹, R² and R³ may have one of the meanings given above, optionally in the form of the tautomers, mixtures of tautomers, hydrates or solvates thereof, and optionally in admixture with one or more physiologically acceptable excipients.

9. Inhalable powders according to claim 1, characterised in that the compounds of general formula 1 have the parameters of a characteristic particle size X₅₀ of 0.1 μm to 10 μm and Q_{(5.8)} of more than 60%.

10. Inhalable powders according to claim 1, characterised in that the compounds of general formula 1 are micronised and then conditioned using an organic solvent selected from among pentane, hexane, heptane, cyclohexane, ethanol, methanol, chloroform and methylene chloride.

11. Inhalable powders according to claim 1, characterised in that they contain only one or more compounds of general formula 1.

12. Inhalable powders according to claim 1, characterised in that they contain, in addition to one or more compounds of general formula 1, at least one physiologically acceptable excipient, optionally in the form of the hydrates or anhydrates thereof.

13. Inhalable powders according to claim 12, characterised in that the physiologically acceptable excipient is selected from the group consisting of monosaccharides, disaccharides, oligo- and polysaccharides, polylactide/glycolide, polyalcohols, amino acids, chitosan, alkali metal and alkaline earth metal salts of stearic acid, salts and mixtures of these excipients with one another, optionally in the form of the hydrates or anhydrates thereof.

14. Inhalable powders according to claim 13, characterised in that the physiologically acceptable excipient is selected from the group comprising glucose, fructose, arabinose, lactose, saccharose, maltose, trehalose, maltodextrin, starch, cellulose, resomer, sorbitol, mannitol, xylitol, arginine hydrochloride, Mg-stearate, sodium chloride, calcium carbonate and mixtures of these excipients with one another, optionally in the form of the hydrates or anhydrates thereof.

15. Inhalable powders according to claim 14, characterised in that the physiologically acceptable excipient is selected from the group comprising glucose, fructose, arabinose, lactose, saccharose, maltose, trehalose, maltodextrin and mixtures of these excipients with one another, optionally in the form of the hydrates or anhydrates thereof.

16. Inhalable powders according to claim 1, characterised in that the ratio of compound of formula 1 to physiologically acceptable excipient is within the range from 5:100 to 1:100000, preferably 1:1000 to 3:1000.

17. Inhalable powders according to claim 1, characterised in that the physiologically acceptable excipient has an average particle size of about 17-120 μm, preferably about 17-90 μm.

18. Method for the treatment of respiratory complaints comprising administering to a patient an inhalable powder according to claim 1.

19. Pharmaceutical formulation, characterised in that it contains an inhalable powder according to claim 1.
